(19) United States
(12) Patent Application Publication
(10) Pub. No.: US 2015/0290045 A1
(43) Pub. Date: Oct. 15, 2015

(54) WOUND DRESSING HAVING GRID PATTERN

(71) Applicants: Ting Fan, Shanghai (CN); Jing Huang, Shanghai (CN); JING Ma, Shanghai (CN); JANE Hu, Shanghai (CN)

(72) Inventors: Ting Fan, Shanghai (CN); Jing Huang, Shanghai (CN); JING Ma, Shanghai (CN); JANE Hu, Shanghai (CN)

(73) Assignee: 3M INNOVATIVE PROPERTIES COMPANY, SAINT PAUL, MN (US)

(21) Appl. No.: 14/235,096

(22) PCT Filed: Jul. 28, 2011
(86) PCT No.: PCT/CN2011/077709

§ 371 Date: Mar. 14, 2014

(51) Int. Cl. A61F 13/02

(52) U.S. Cl. CPC A61F 13/0243; A61F 13/0246; A61F 13/0289

(57) ABSTRACT

A wound dressing comprising a base film layer which is made of polymer and have a grid pattern on one surface of the substrate layer is provided. A method of making the wound dressing comprising the steps of coating a polymer solution on a patterned liner and drying the polymer solution is also provided.

### FIELD OF THE DISCLOSURE

The present disclosure relates to a wound dressing having a grid pattern on its surface and a method of making the wound dressing.

### BACKGROUND

To measure the size of a wound is critical in documenting the progress of healing and in assessing the effectiveness of interventions on the healing processes in clinical and research settings. A wound dressing having a printed grid on the top surface of the dressing are used clinically. A grid made by printing tends to disappear over time or from rubbing, and therefore it becomes difficult to measure the wound accurately.

It is desired to produce a wound dressing which has a stable and accurate grid and is able to easily and accurately measure the wound size in various conditions.

### SUMMARY

The present disclosure provides a wound dressing comprising a base film layer which comprises a polymer film having a grid pattern on one surface of the base film layer, and an adhesive layer or a hydrocolloid layer disposed on the opposite surface of the base film layer from the surface having the grid pattern, wherein the grid pattern of the polymer film is obtained by coating a polymer solution on a patterned liner having a complementary pattern to the grid pattern of the polymer film. The present disclosure also provides a method of making the wound dressing comprising the steps of coating a polymer solution on the patterned liner, and drying the polymer solution and replicating the grid pattern of the patterned liner to the surface of the base film layer.

### BRIEF DESCRIPTION OF THE DRAWINGS

FIGS. 1a and 1b are sectional views of the wound dressing of the present disclosure.

FIG. 2 is a top view of the wound dressing of the present disclosure.

FIGS. 3a to 3c are sectional views of the wound dressing according to a further embodiment of the present disclosure.

FIG. 4 depicts a process of making the wound dressing of the present disclosure.

### DETAILED DESCRIPTION

The wound dressing of the present disclosure comprises a base film layer and an adhesive layer or a hydrocolloid layer.

The base film layer comprises a polymer film which has a grid pattern on one surface. A wound on the skin such as, for example, a bedsore or pressure ulcer can be measured with the grid of the wound dressing when the dressing is applied on the skin covering the wound area.

The adhesive layer or the hydrocolloid layer is disposed on the opposite surface of the base film layer from the surface having the grid pattern. As used herein “grid pattern” means a recessed or protruded structure on the surface of the base film layer. The grid pattern is obtained by coating a polymer solution to a patterned liner having a complementary pattern to the grid pattern of the polymer film.

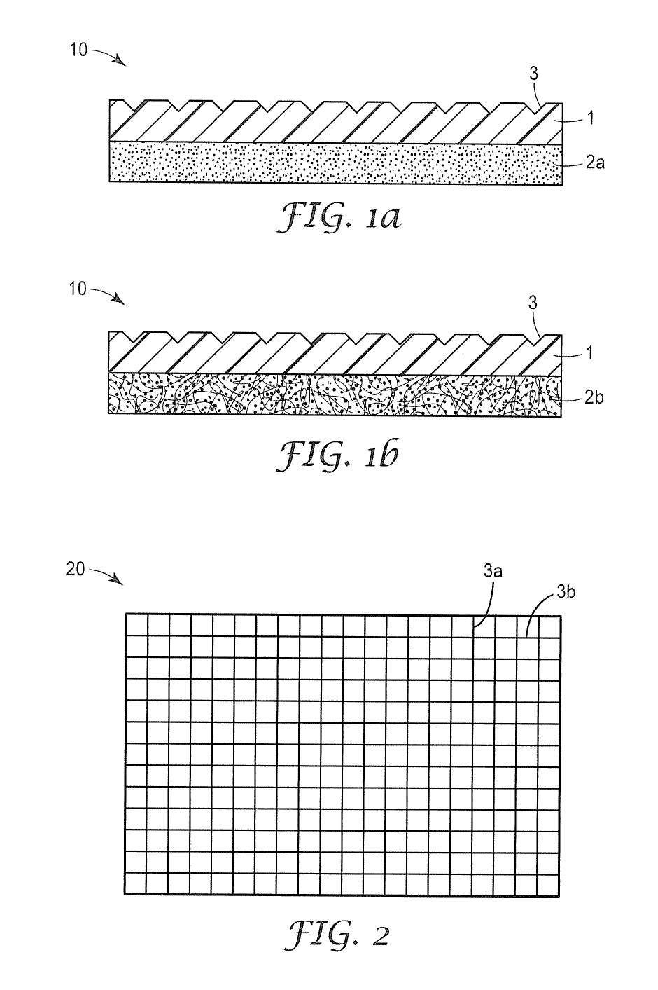

FIGS. 1a and 1b depict a cross sectional views of the wound dressing of the present disclosure. FIG. 1a shows the wound dressing comprising a base film layer 1 having a recessed grid pattern 3 and an adhesive layer 2a. FIG. 1b shows the wound dressing comprising the base film layer 1 having the recessed grid pattern 3 and a hydrocolloid layer 2b.

The base film layer 1 comprises a polymer film. The polymer film comprises a polymer which may be preferably flexible to conform to the skin and transparent to allow the wound to show. Examples of the polymer include polyurethanes; polyolefins such as polyester, polypropylene and polybutylene; polyesters such as polyethylenetelephtalate (PET); fluorinated polymers such as polytetrafluoroethylene (PTFE) and polyvinylidene fluoride (PVDF); nylon and polyvinylchloride (PVC).

The thickness of the base film layer is not limited, but may be from about 4 micrometers to about 100 micrometers, or about 6 micrometers to about 80 micrometers.

The recessed grid pattern 3 is on one surface of the base film layer. The surface may be the outermost surface of the wound dressing. The recessed grid pattern 3 is obtained by coating a polymer solution constituting of the base film layer to a patterned liner having a complementary protruded pattern to the recessed grid pattern 3.

The patterned liner is a liner having a recessed or protruded structure on the surface and may be made by bare or coated polymer or paper. Example of the patterned liner includes Comply Liner (available from 3M Company, St. Paul, Minn., USA). The patterned liner is used for making the base film layer having the grid pattern. The patterned liner may be peeled off from the wound dressing when the wound dressing is applied to the skin.

The adhesive layer 2a may include known pressure sensitive adhesive such as for example rubber adhesive, acrylic adhesive, polyester adhesive or silicone adhesive.

The hydrocolloid layer 2b may include known hydrocolloid material which comprises hydrocolloid absorbent such as celloluse gum, hydrophobic unsaturated elastomeric homoplomer and polyisobutylene tackifier. The hydrocolloid material may further include resin tackifier. The specific examples of the hydrocolloid layer 2b include 3M™ Tegaderm™ Hydrocloid Dresssing (available from 3M Company, St. Paul, Minn., USA), Coloplast Comfeel™ Ulcer Dressing (available from Coloplast Limited, Peterborough, Cambs, England) and ConvaTec Duoderm™ CGF Hydrocolloid Dressing (available from ConvaTec Inc., Skillman, N.J., USA).

The adhesive layer 2a and the hydrocolloid layer 2b may be made by known method such as for example coating or extruding the above mentioned adhesives onto the base film layer or laminating an adhesive sheet comprising the above mentioned adhesives to the base film layer. The known coating process such as bar coating and knife coating or known extruding process can be used respectively for coating or extruding the adhesive or the hydrocolloid material.

The thickness of the adhesive layer or the hydrocolloid layer is not limited, but may be about 4 micrometers to about 5000 micrometers or about 10 micrometers to about 3000 micrometers.

The wound dressing may further include a release liner disposed on the adhesive layer. The release liner can protect the surface of the adhesive layer or the hydrocolloid layer and peel off from the wound dressing when applied to the skin.

In one embodiment, the grid pattern includes a vertical grid line and a horizontal grid line and those lines are at substantially right angles to one another. FIG. 2 depicts the top view of the wound dressing of the present disclosure. The grid pattern has a vertical grid line 3a and a horizontal grid line 3b. The interval between the neighboring lines may be preferably equal. The vertical grid line 3a and the horizontal grid line 3b may be preferably at substantially right angles to one another. The distance between neighboring grid lines is not limited, but may be from about 1 mm to about 50 mm or about 2 mm to about 20 mm, more preferably it may be about 2 mm, about 5 mm or about 10 mm.

In one embodiment, the grid pattern includes concentric geometric shapes. In one embodiment, the grid pattern includes concentric geometric shapes with radially extending lines. For example, the geometric shapes may be circles, ovals, triangles, rectangles or squares.

The wound size on the skin can be observed and measured with the grid pattern. Specifically, the wound size can be measured as the numbers of the vertical grid lines for a cross direction by the numbers of the horizontal grid lines for a longitudinal direction such as for example, 24 grids by 15 grids or 30 grids by 7 grids. The process of healing wound can be observed as a size reduction of the wound.

The depth or height of the grid pattern may be set depending on the height or depth of the pattern of the patterned liner and is not limited, but may be from about 1 micrometer to about 90 micrometers or from about 2 micrometers to about 80 micrometers.

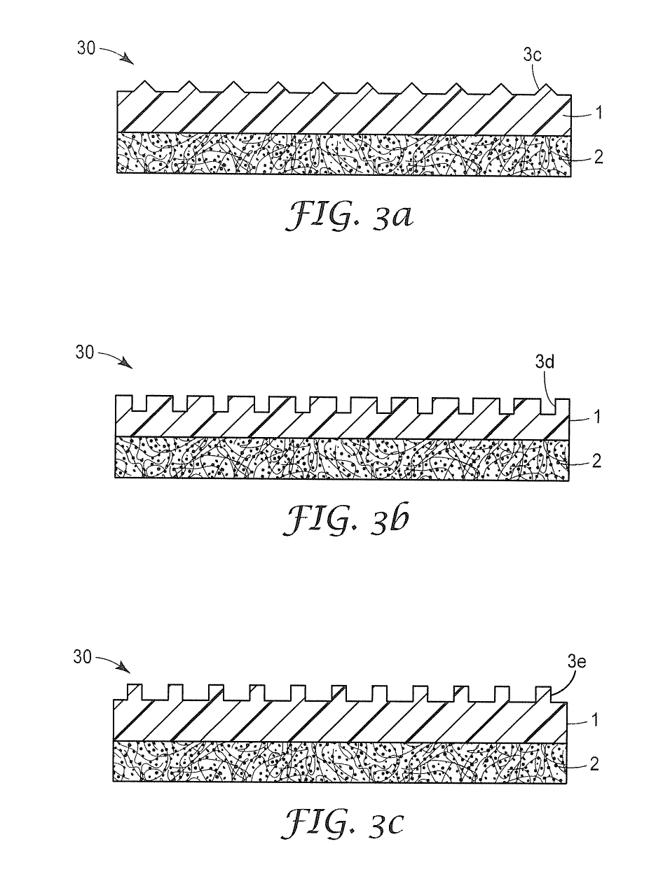

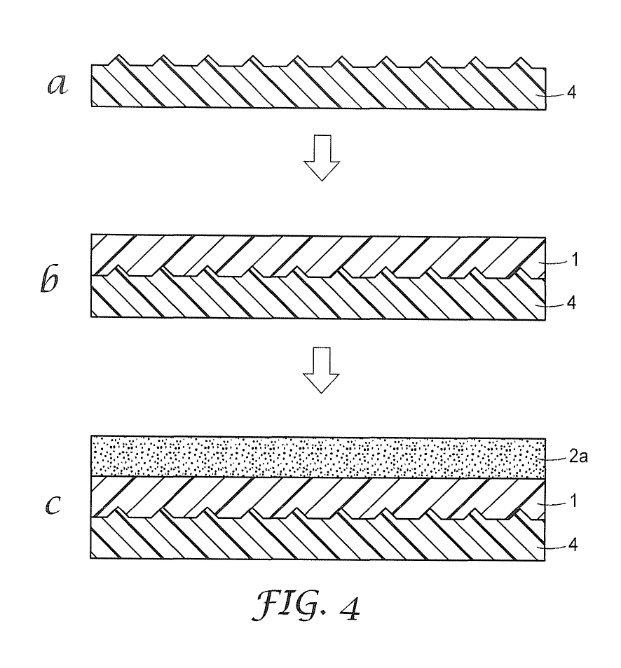

FIGS. 3a to 3c depict the cross sectional view of the wound dressing of further embodiments of the present disclosure. FIGS. 3a to 3c show examples of the shape of the grid pattern. FIG. 3a shows the wound dressing which has a protruded grid pattern 3 having a pointy shape. FIG. 3b shows the wound dressing which has a recessed grid pattern 3 having a square shape. FIG. 3c shows the wound dressing which has a protruded grid pattern 3 having a square shape. The grid patterns may be made by coating a polymer on the patterned liner having the complementary pattern to the desired grid pattern.

The wound dressing may be prepared by known methods such as coating a polymer solution on the patterned liner to form a base film layer and then coating or laminating an adhesive layer onto the obtained base film layer. FIG. 4 shows the process of making the wound dressing of the present disclosure. Referring to FIG. 4, a patterned liner 4 having a grid pattern on its surface is prepared (FIG. 4-a), then a polymer solution is coated by a known coating method on the surface of the patterned liner 4 and dried under heating to form the base film layer 1 (FIG. 4-b), which has a complementary grid pattern to the pattern of the patterned liner. Then the adhesive layer 2 is laminated on the opposite surface of the obtained base film layer 1 from the patterned liner 4 to give the wound dressing (FIG. 4-c). Alternatively, the adhesive is coated by a known coating method onto the obtained base film layer and dried with an oven to form the adhesive layer, and to give the wound dressing.

The wound dressing of the present disclosure may be applied on the skin having a wound and measure the size of the wound, then the progress of healing may be documented easily. Further the grid pattern of the wound dressing is made by coating the polymer to the patterned liner, so it can be stable and kept in a good condition and the progress of healing may be documented more accurately.

The adhesive layer or the hydrocolloid layer may further contain functional medicines such as antimicrobial drug, antifungal drug or antibiotic drug for treating a wound.

### EXAMPLES

### Example 1

### Wound Dressing (Polyurethane (PU) Film and Adhesive Layer)

This example shows the preparation of the wound dressing comprising a PU film (base film layer) and an adhesive layer as described in FIG. 1(a).

A PU solution (V-5454LV available from Jiaxing Puyou, Zhejiang, China) was coated on a liner (Comply liner CB1 available from 3M Company, St. Paul, Minn., USA) with a bar coater, dried for five minutes at 130 degrees C. with an oven and gave the PU film. The Comply liner CB1 had a grid pattern on its surface and the distance between the grids was 0.5 mm. The thickness of the obtained PIJ film was 20 micrometers (dry) and the depth of grid was 10 micrometers.

A water base adhesive comprising 50% acrylic polymer (available from 3M Company, St. Paul, Minn., USA) was coated on the PU film directly with a bar coater, then dried for five minutes under 110 degrees C. with an oven and gave the wound dressing.

### Example 2

### Wound Dressing (PU Film and Hydrocolloid Layer)

This example shows the preparation of the wound dressing comprising a PU film (base film layer) and hydrocolloid layer as described in FIG. 1(b).

PU film was prepared by the same process as Example 1. The obtained PU film was laminated with a hydrocolloid sheet (450 micrometers thick, available from 3M Company, St. Paul, Minn., USA) to produce the wound dressing.

1-5. (canceled)

6. A wound dressing comprising:
a base film layer which comprises a polymer film having a grid pattern on one surface of the base film layer, and
an adhesive layer or a hydrocolloid layer disposed on the opposite surface of the base film layer from the surface having the grid pattern,
wherein the grid pattern of the polymer film is obtained by coating a polymer solution to a patterned liner having a complementary pattern to the grid pattern of the polymer film.

7. The wound dressing according to claim 6, wherein the polymer film is transparent.

8. The wound dressing according to claim 6, wherein the grid pattern has a vertical grid line and a horizontal grid line, and the vertical and horizontal grid lines are at right angle to one another.

9. The wound dressing according to claim 6 further comprising a release liner on the opposite surface of the adhesive layer or the hydrocolloid layer from the base film layer.

10. A method of making the wound dressing according to claim 6 comprising the steps of:
coating a polymer solution on the patterned liner, and
drying the polymer solution and replicating the grid pattern of the patterned liner to the surface of the base film layer.
